(19) United States
(12) Patent Application Publication
(10) Pub. No.: US 2025/0213270 A1
(43) Pub. Date: Jul. 3, 2025

(54) STABILIZING TISSUE ACCESS NEEDLE DEVICES, SYSTEMS AND METHODS

(71) Applicant: SureAx Medical Corp., San Diego, CA (US)

(72) Inventors: Greg Kelley, San Diego, CA (US); Ghassan Kassab, La Jolla, CA (US); Neil Drake, San Diego, CA (US)

(73) Assignee: SureAx Medical Corp., San Diego, CA (US)

(21) Appl. No.: 18/850,502

(22) PCT Filed: Mar. 24, 2023
(86) PCT No.: PCT/US23/16327

§ 371 Date: Sep. 24, 2024

Related U.S. Application Data
(63) Continuation-in-part of application No. 17/794,849, filed on Jul. 22, 2022.
(60) Provisional application No. 63/323,490, filed on Mar. 24, 2022.
(60) Provisional application No. 62/965,159, filed on Jan. 23, 2020.

(51) Int. Cl. A61B 17/34

(52) U.S. Cl. CPC A61B 17/34; A61B 2017/3488

(57) ABSTRACT

Needle devices for accessing tissue at a desired location within a patient's body in order to deliver biologic, chemical or other therapeutic agents to the tissue, or obtain a sample of the tissue, or to obtain a sample of body fluid or otherwise drain body fluid from the body of a patient, and secured after access to the tissue node is described. The needle device includes a hub, a needle and a stabilizing wire. Securing the needle can be accomplished via stabilizing wire with a helical tissue engagement portion, which can be introduced via an open needle tip or a lumen aperture. The stabilizing wire may be attached to a slidable trigger on the hub. Fluid can be introduced into the needle device through an injection port in the hub with an extension element thereon.

### PRIORITY

The present patent application is related to, and claims the priority benefit of, U.S. Provisional Patent Application Ser. No. 63/323,490, filed on Mar. 24, 2022, the contents of which are hereby incorporated by reference in their entirety into this disclosure.

### BACKGROUND

There are a variety of medical procedures using needles to access tissue at a desired location within a patient's body in order to deliver biologic, chemical or other therapeutic agents to the tissue, or obtain a sample of the tissue, or to obtain a sample of body fluid or otherwise drain body fluid from the body of a patient. Optimal position and stability of the needle at a desired location within a patient's body during the procedure is often critical.

For example, intranodal lymphangiography (IL) is the leading method of imaging of the lymphatic system worldwide. IL is performed by accessing the lymph nodes with a small gauge needle using ultrasound guidance (USG). A contrast material is then slowly injected into the lymph node, and the lymphatic system is then imaged. However, there are limitations with the IL procedure such as positioning of the needle precisely in the central part of the lymph node and securing the needle there during the procedure or patient transfer leading to frequent dislodgement of the needle, and greatly impairing the quality of the imaging and success of the procedure.

Another area where optimal position and stability of the needle is neurological/spinal procedures. For example, in occipital nerve stimulation, a doctor uses a needle to place stimulating electrodes at the base of the skull with leads connected to a power source (pulse generator) that sends electrical impulses to the occipital nerve. If the needle is advanced too deep, or if the needle is easily dislodged and moves around, it can injure the brain stem.

Other procedures where optimal position and stability of a needle is critical include the delivery and placement of a peripherally inserted central catheter (PICC), chemotherapy, biopsies, gene therapy, chimeric antigen receptor T cell (CAR T cell) therapy, and ascites diagnosis and/or treatment.

There are a variety of needles known in the art that can be secured in tissue at a desired location within a patient's body. However, current needles often require significant manual manipulation by a doctor performing the procedures, or even require assistance from an additional person, to secure the needle while simultaneously delivering an agent, or obtaining a sample of tissue or fluid with the needle. Thus, there is a need in the art for a simplified needle, device or system allowing a physician to more easily secure the needle in tissue at a desired location within a patient's body.

### BRIEF SUMMARY

The present disclosure includes disclosure of a needle device, comprising a needle having a needle lumen defined therethrough terminating at a needle aperture at or near a distal end of the needle, the needle device configured to engage a tissue and further configured to introduce a fluid through the needle lumen into the tissue or a lumen thereof. The present disclosure includes disclosure of a needle device, further comprising a stabilizing wire positioned within the needle lumen, the stabilizing wire configured to at least partially protrude from the needle aperture and to engage a tissue. The present disclosure includes disclosure of a needle device, wherein the stabilizing wire comprises a helical portion with a sharp tip, and wherein the helical portion is configured to engage a tissue.

The present disclosure includes disclosure of a needle device, further comprising a hub configured to deploy the stabilizing wire from the needle aperture and to retract the stabilizing wire into the needle aperture. The present disclosure includes disclosure of a needle device, wherein the hub comprises a slide mechanism operably connected to the stabilizing wire to propel the stabilizing wire into the tissue. The present disclosure includes disclosure of a needle device, wherein the slide mechanism is configured to be manually triggered. The present disclosure includes disclosure of a needle device, wherein the stabilizing wire comprises a helical distal end terminating with a sharp and distal tip.

The present disclosure includes disclosure of a needle device, comprising multiple lumens defined therethrough terminating at a needle aperture at or near a distal end of the needle, and a stabilizing wire positioned within a first of the multiple lumens, wherein the stabilizing wire comprises a helical portion, and wherein the helical portion is configured to at least partially protrude from the needle aperture and to reversibly engage a tissue, the needle device configured to reversibly puncture a tissue and further configured to introduce a fluid through a second of the multiple lumens into the tissue or a lumen thereof.

The present disclosure includes disclosure of a needle device, comprising a lumen defined therethrough terminating at a needle aperture at or near a distal end of the needle, and multiple stabilizing wires positioned within the lumen, wherein the stabilizing wires each comprise a helical portion, and wherein the helical portion is configured to at least partially protrude from the needle aperture and to reversibly engage a tissue, the needle device configured to reversibly puncture a tissue and further configured to introduce a fluid through the lumen into the tissue or a lumen thereof.

The present disclosure includes disclosure of a method for using a needle device to access tissue at a desired location within a patient's body, the method comprising the steps of inserting a needle into the tissue of a patient, wherein the needle comprises a lumen and a needle aperture; deploying a stabilizing wire to attach the needle device to the tissue; introducing fluid into the tissue; retracting the stabilizing wire; and removing the needle. The present disclosure includes disclosure of a method, wherein the step of introducing fluid comprises the step of introducing fluid through an injection port in a hub having an extension member thereon.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a device for stabilized accessing of tissue at a desired location within a patient's body including a needle with stabilizing wire and a hub with an extension set, according to an embodiment of the present disclosure;

FIG. 2 shows a device for stabilized accessing of tissue at a desired location within a patient's body including a needle with stabilizing wire and a hub with an extension set, according to an embodiment of the present disclosure;

FIG. 3 shows a needle according to an embodiment of the present disclosure;

FIG. 4A shows a stabilizing wire to be used with a needle according to an embodiment of the present disclosure;

FIG. 4B shows a stabilizing wire to be used with a needle according to an embodiment of the present disclosure;

FIG. 4C shows a stabilizing wire to be used with a needle according to an embodiment of the present disclosure;

FIG. 5 shows a needle device accessing tissue at a desired location within a patient's body with the stabilizing wire deployed to secure the needle according to an embodiment of the present disclosure;

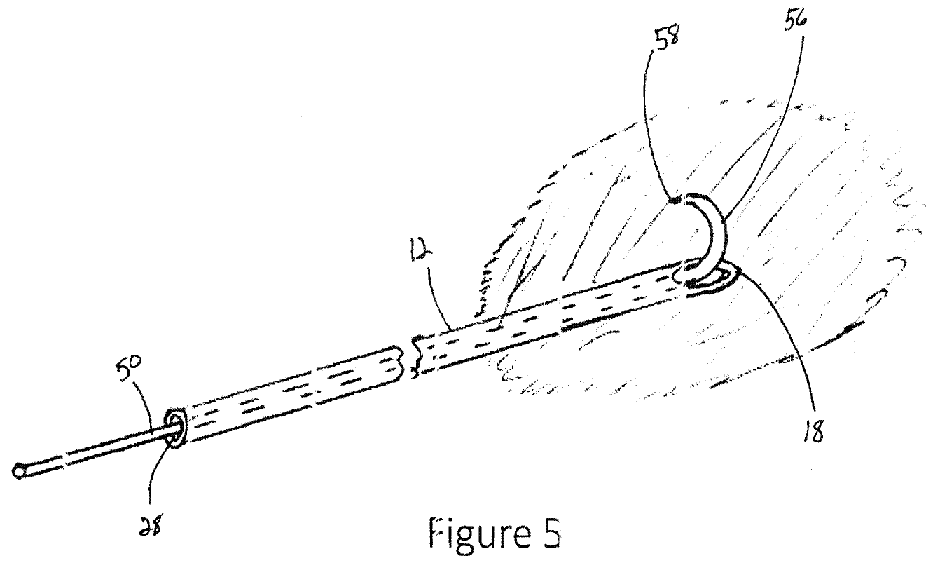

FIG. 6 shows a needle device accessing tissue at a desired location within a patient's body with the stabilizing wire retracted according to an embodiment of the present disclosure;

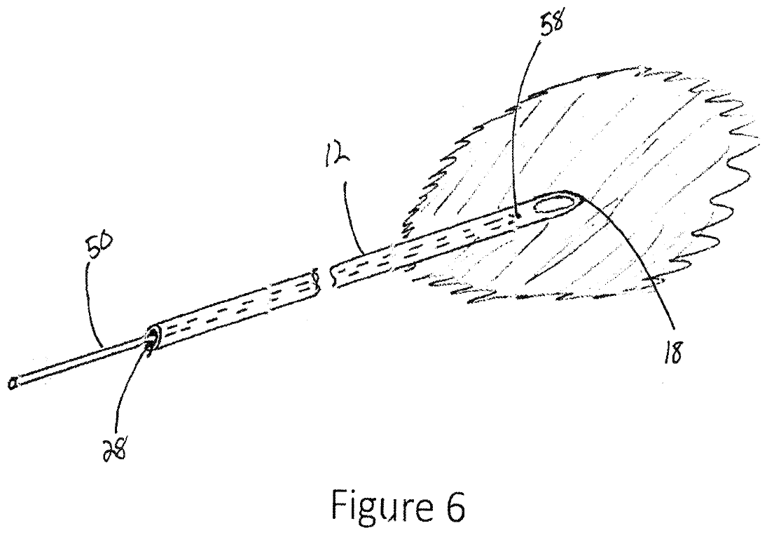

FIG. 7 shows a hidden, dashed-line view of a multi-lumen needle device accessing tissue at a desired location within a patient's body with the stabilizing wire deployed proximal of the tissue at the desired location, according to an embodiment of the present disclosure;

FIG. 8 shows a sectional view of a multi-lumen needle device accessing tissue at a desired location within a patient's body with the stabilizing wire deployed proximal of the tissue at the desired location, according to an embodiment of the present disclosure;

FIG. 9 shows a hidden, dashed-line view of a multi-lumen needle device accessing tissue at a desired location within a patient's body with the stabilizing wire deployed distal of the tissue at the desired location, according to an embodiment of the present disclosure;

FIG. 10 shows a needle device with multiple stabilizing wires deployed, according to an embodiment of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

In the present disclosure, the term “distal” is used to refer to the part of the device farthest from an operator or closest to a patient. The term “proximal” is used to describe the part of the device closest to an operator or farthest from a patient. For example, the part of the needle that is inserted into the patient first will be the distal part.

As set forth above, the present invention is suitable for use in accessing a target site within a body. A target site can be located in any anatomic location in the body. Typically a site is identified by the physician or radiologist and the tissue, cells and/or fluid, or other material at that site, is then identified as target material and selected or targeted for access. Thus, for example, the target tissue, cells or fluid can be the target material identified for access from: lymph, brain, heart, liver, kidney, thyroid, lung, pancreas, intestine, uterine, ovary, prostate, spleen, skin, biliary, parathyroid, pituitary, adrenal gland, mediastinum, bladder, connective tissue, breast, gastrointestinal tract, joints, muscle, etc. Additionally, in some instances it is desirable to access a target site located within a void, such as a space between organs, lumen, etc. In that instance, the target site may include fluid, or other material which is the target for access. Once the target site is accessed and target material (e.g. tissue, cells and/or fluid) is identified for access, one or more diagnostic, therapeutic or delivery procedures can be employed to remove, treat and/or mark the target material.

Although the device can be used in a variety of procedures, for the purposes of promoting an understanding of the principles of the present disclosure, the device and its operation will be discussed in the context of lymphatic access, and reference will now be made to the embodiments illustrated in the drawings, and specific language will be used to describe the same. It will nevertheless be understood that no limitation of the scope of this disclosure is thereby intended.

An application of the device includes intranodal lymphangiogram during which the distal end of a needle is positioned inside a lymph node under ultrasound guidance and contrast is slowly injected through the needle into the lymph node, wherein the needle tip is secured to the lymph node to prevent dislodgment of the needle from the lymph node to ensure no contrast is injected outside of the lymph node.

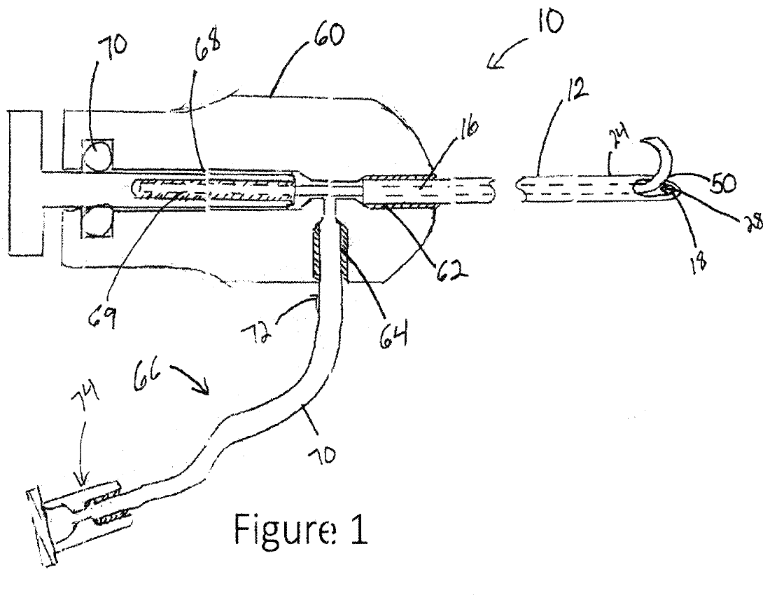

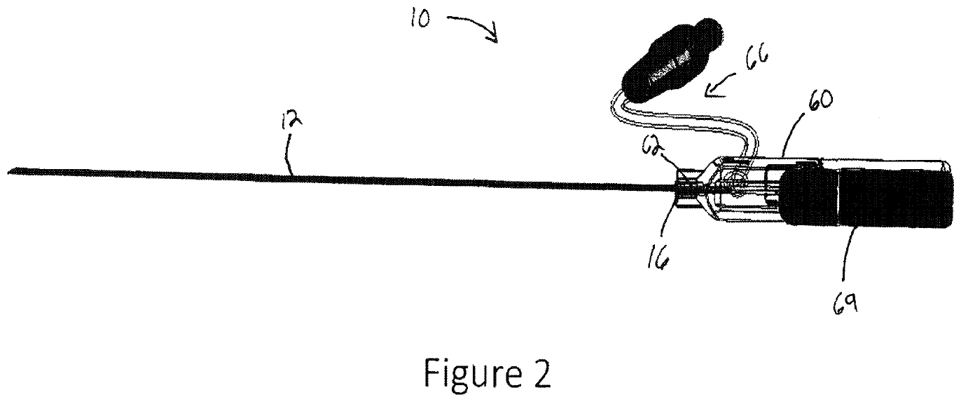

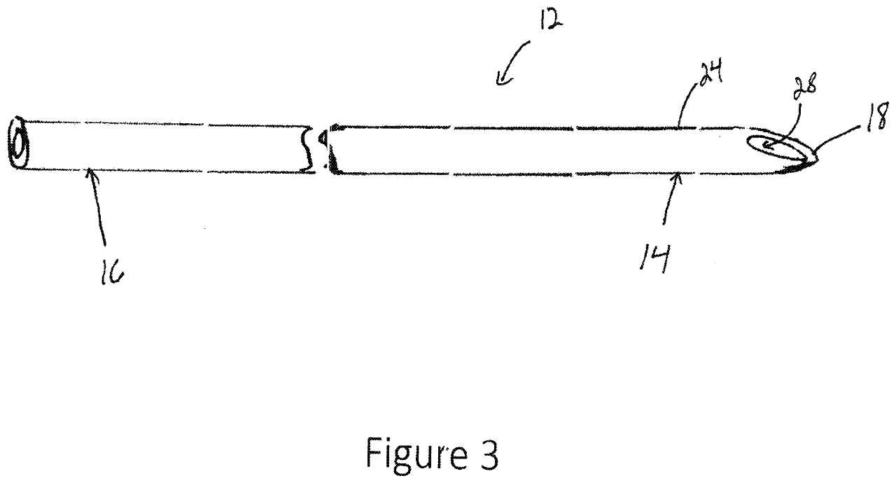

One embodiment of a needle device 10 of the present invention shown in FIGS. 1-2 comprises a needle 12, a stabilizing wire 50, and a hub 60. As shown in FIGS. 1-3. The needle 12 comprises a distal end 14 comprising an open distal tip 18, a proximal end 16, and a sidewall 24 defining a needle lumen 28. The open distal tip 18 of needle 12 is sharp to aid in puncturing tissue. The open distal tip 18 may also be configured to be echogenic. The hub 60 includes a needle port 62, an injection port 64 with extension set 66, a stabilizer port 68, and a seal 70. The proximal end 16 of needle 12 is connected to the needle port 62 of the hub 60. The needle device 10 is configured to allow fluid communication between the injection port 64 and the needle port 62 and the needle lumen via the proximal end 16 of the needle 12 positioned within the needle port 62. For example, contrast media can be introduced into the injection port 64 via a syringe or other method connected to the extension set 66, and then travel to the needle port 62, into the open proximal end 16 of the needle 12, and then through the needle lumen 28 to exit the needle 12 at the open distal tip 18.

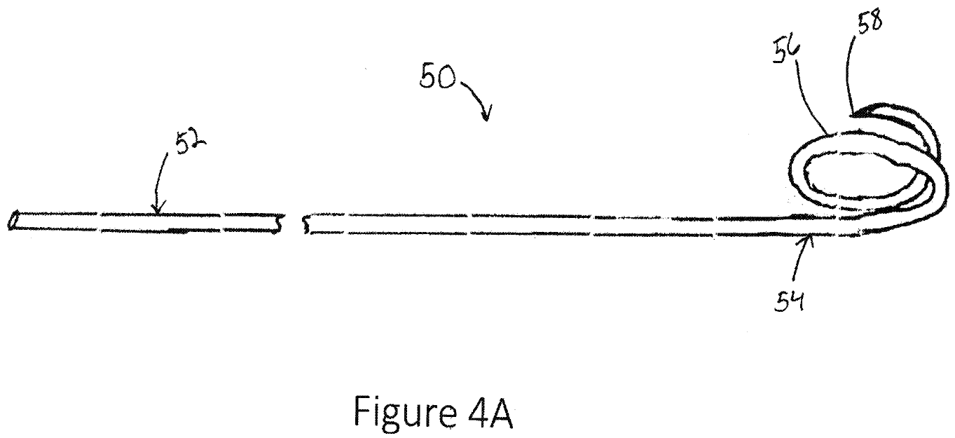

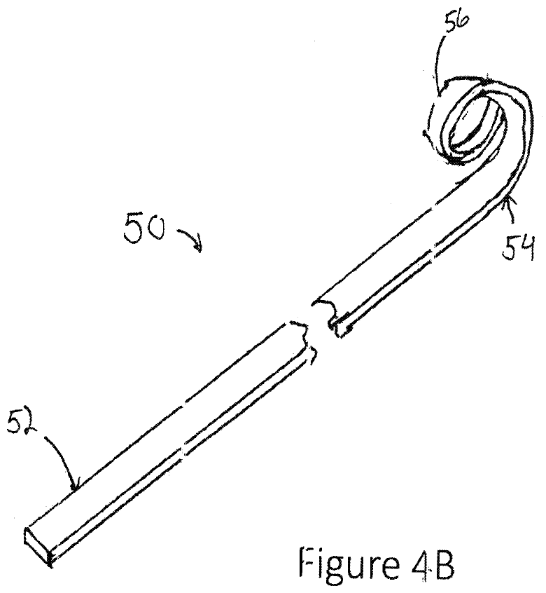

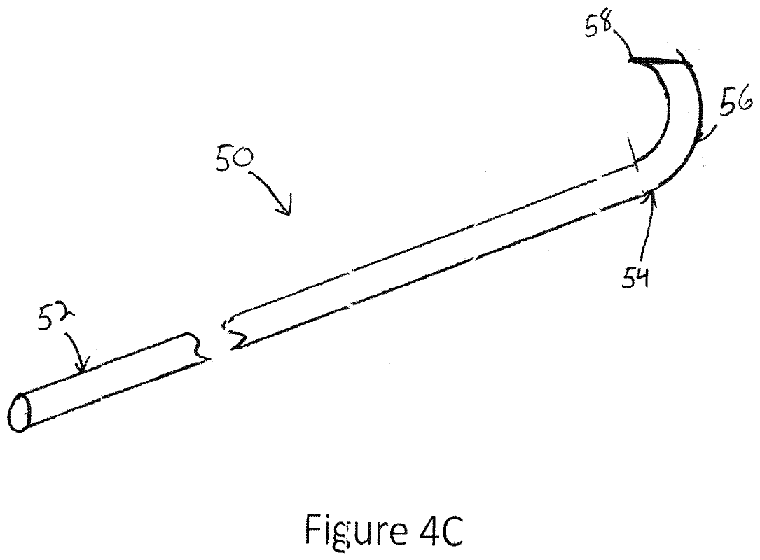

As shown in FIGS. 4A-4C, the stabilizing wire 50 comprises a proximal end 52 and a distal end 54. The distal end 54 comprises a helical portion 56 with a sharp tip 58. The stabilizing wire 50 is slidingly disposed within the needle lumen 28, the proximal end 52 being positioned within the stabilizer port 68 of the needle hub 60. The stabilizing wire 50 is operably connected to a slide trigger 69 on the hub 60. The slide trigger 69 is configured such that it can be moved longitudinally on an exterior surface of the hub 60, thereby causing the stabilizing wire 50 to slide longitudinally within the stabilizer port 68 of hub 60. The slide trigger 69 is configured to slide a sufficient distance back and forth along a longitudinal axis of the hub 60 such that it causes the helical portion 56 of the stabilizer wire 50 to slide between a retracted position and a deployed position. For example, when the slide trigger 69 is moved to pull the stabilizing wire 50 to a proximal position within the stabilizer port 68 of the needle hub 60, the stabilizing wire 50 is pulled into a retracted position wherein the helical portion 56 is pulled into the needle lumen and whereby the helical portion 56 is substantially straightened and constrained within the needle lumen 28. When the slide trigger 69 is moved to push the stabilizing wire 50 to a distal position within the stabilizer port 68 of the needle hub 60, the sharp tip 58 at the end of the helical portion 56 of the stabilizing wire 50 is pushed out through the open distal tip 18 of the needle 12 and deployed into surrounding tissue.

The stabilizing wire 50 may be made from a material with relatively high elastic deformation, such as nitinol or similar materials, such that the helical portion 56 of the stabilizing wire 50 can recover its shape as it is being deployed from the distal tip 18 of the needle 12 after being substantially straightened while retracted into the needle lumen 28. The stabilizer wire may be made from round or flat wire. Thinner flat wire allows the helical portion 56 to be formed to a smaller radius and still recover its helical shape after being substantially straightened within the needle lumen 28. Thus, as the stabilizing wire 50 exits the needle lumen 28, the helical portion 56 recovers its helical shape, and the sharp tip 58 advances along the path of the helix and penetrates surrounding body tissue near the distal tip 18 of the needle 12 thereby securing the needle 12 to the tissue. The shape of the helical portion 56 reduces penetration resistance, and also helps the stabilizing wire 50 to pull itself out of the needle lumen 28, and the sharp tip 58 makes for easier penetration and also enhances inward deflection. The length of the helical portion 56 of stabilizing wire 50 depends upon the requirements of the particular application for which the needle device 10 is being used.

As described above, and as apparent to one of ordinary skill in the art, the constituent elements can be interchanged to produce a device for particular use in accessing and securing to lymph nodes. Some of the interchangeability is discussed further below.

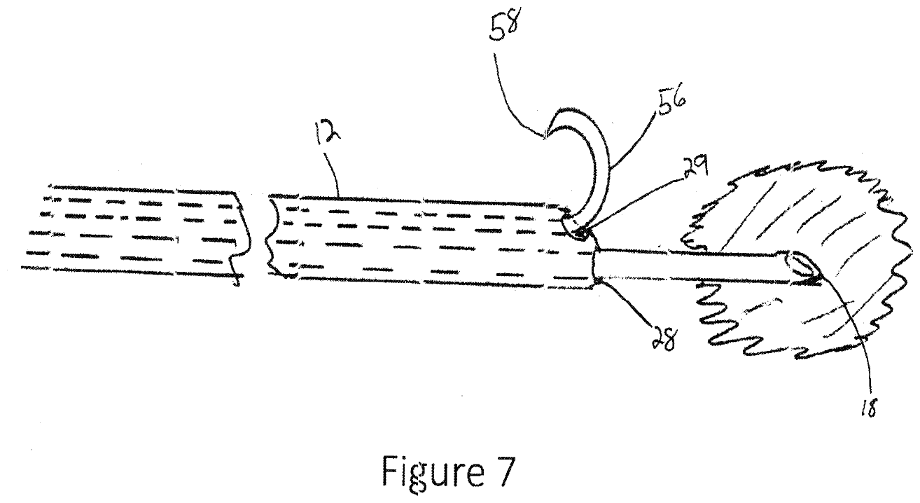

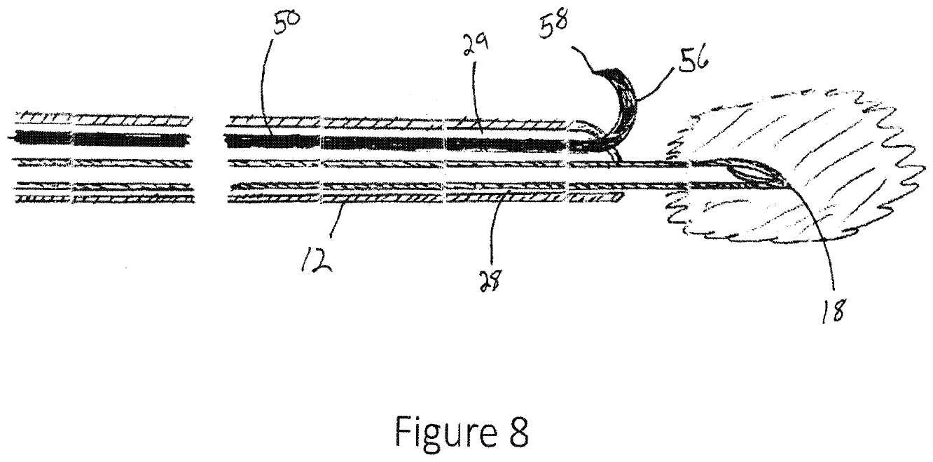

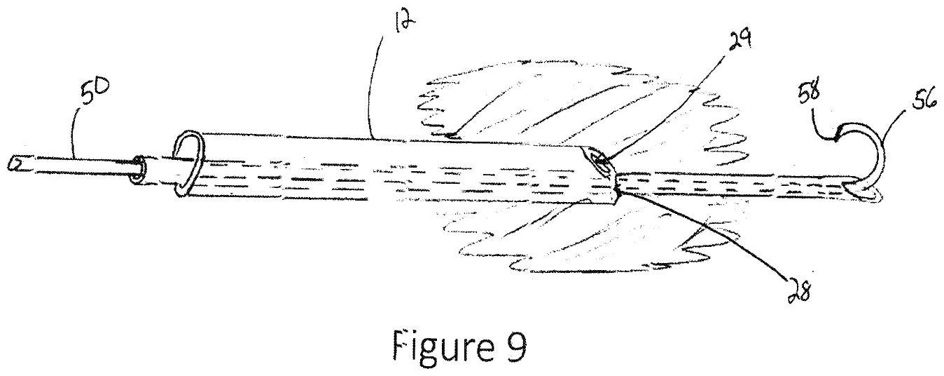

For example, FIGS. 7-9 show an embodiment of a needle device 10 wherein the needle 12 comprises multiple lumen 28, 29, and the stabilizing wire 50 may be slidably disposed in either lumen 28 or 29, and injection of a contrast media, or other type of agent, can be delivered through the other of lumen 28 or 29. In the embodiment depicted in FIGS. 7-8, needle 12 is configured such that stabilizing wire 50 is deployed through lumen 29 at a point proximal to the target tissue and lumen 28 is positioned in the target tissue whereby a therapeutic agent is delivered through lumen 28 to the target tissue. In the embodiment in FIG. 9, needle 12 is configured such that stabilizing wire 50 is deployed through lumen 28 at a point distal to the target tissue and lumen 29 is positioned in the target tissue whereby a therapeutic agent is delivered through lumen 29 to the target tissue.

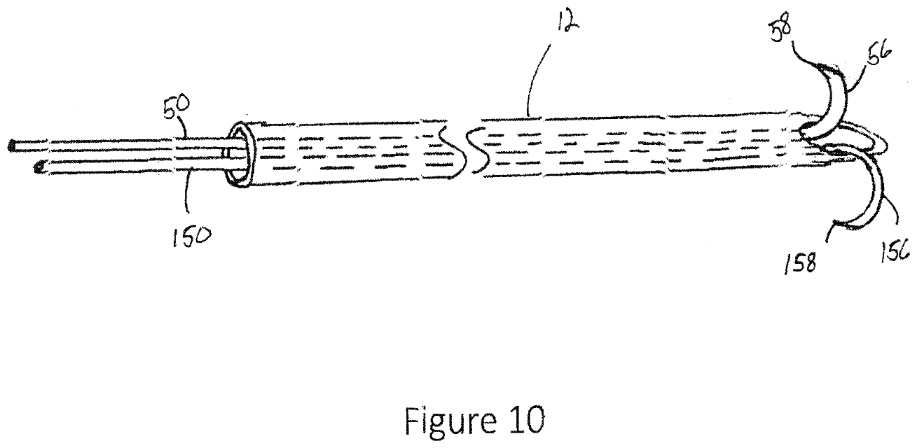

FIG. 10 shows an embodiment in which multiple stabilizing wires 50, 150 are disposed in lumen 28 of needle 12.

Also a curved needle can be used with the invention of the present disclosure.

In operation a user, such as a physician, places the needle 12 into the tissue at the desired location within a patient's body. Once the needle 12 has been positioned at the desired location, the physician slides the trigger 69 on the hub 60 to deploy the stabilizing wire 50. As the stabilizing wire 50 exits the needle lumen 28, the helical portion 56 of the stabilizing wire 50 recovers its helical shape, and the sharp tip 58 advances along the path of the helix and penetrates surrounding body tissue near the distal tip 18 of the needle 12 thereby securing the needle 12 to the tissue. The stabilizing wire 50 stabilizes the needle 12 against both push and pull forces. Once the stabilizing wire 50 has been deployed and the needle 12 is secured, the physician can inject fluid, such as contrast media, into the injection port 64 of the hub 60 through the extension element 66, which comprises a flexible tube 70 connected to the injection port 64 at a distal end 72, and having a proximal end 74 configured to engage with a syringe or other delivery source as is known in the art. The fluid will then travel into the needle port 62 of the hub 60 and into and through the needle lumen 28 and out the open distal tip 18 of the needle 12 into the tissue. After the fluid has been delivered the physician slides the trigger 69 to retract the stabilizing wire 50 and can remove the needle 12 from the patient.

While various embodiments of devices for accessing tissue at a desired location within a patient's body in order to deliver biologic, chemical or other therapeutic agents to the tissue, or obtain a sample of the tissue, or to obtain a sample of body fluid or otherwise drain body fluid from the body of a patient and methods for the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A needle device, comprising:
a needle having a needle lumen defined therethrough terminating at a needle aperture at or near a distal end of the needle;
at least one stabilizing member having a helical distal end terminating with a sharp distal tip positioned within the needle lumen and configured to slide between a first position and a second position, wherein when the at least one stabilizing member is in the first position, the helical distal end is retracted into the needle lumen such that the helical distal end is substantially straightened, and when the at least one stabilizing member is in the second position, the helical distal end at least partially protrudes from the needle aperture in a helical form whereby it reversibly engages a tissue at or near the distal end of the needle.

2. The needle device of claim 1, further comprising:
a hub having a needle port, an injection port, a stabilizer port and an actuator, wherein a proximal end of the needle is connected to the needle port, the hub being configured to allow fluid communication between the injection port, the needle port and the needle lumen; and
wherein a proximal end of the at least one stabilizing member is positioned in the stabilizer port and operably connected to the actuator, the actuator being configured such that the actuator can be moved longitudinally, thereby causing the at least one stabilizing member to slide longitudinally within the stabilizer port.

3. The needle device of claim 2, further comprising an elongated, flexible extension tube having a distal end connected to the injection port, and a proximal end configured to engage with a syringe.

4. The needle device of claim 1, wherein the at least one stabilizing member is made from nitinol.

5. The needle device of claim 1, wherein the distal end of the needle is configured to be echogenic.

6. The needle device of claim 1, wherein the at least one stabilizing member is made from round or flat wire.

7. The needle device of claim 6, wherein the stabilizing member is made from flat wire.

8. The needle device of claim 1, wherein the helical distal end of the at least one stabilizing member is configured such that when the helical distal end at least partially protrudes from the needle aperture, the sharp distal tip is offset from a longitudinal axis of the needle.

9. The needle device of claim 1, wherein the at least one stabilizing member comprises two or more stabilizing members.

10. A needle device, comprising:
a needle having at least a first lumen and a second lumen defined therethrough, the first lumen terminating at a first aperture at or near a distal end of the needle, and the second lumen terminating at a second aperture at or near the distal end of the needle;
at least one stabilizing member having a helical distal end terminating with a sharp distal tip positioned within the second lumen and configured to slide between a first position and a second position, wherein when the at least one stabilizing member is in the first position, the helical distal end is retracted into the second lumen such that the helical distal end is substantially straightened, and when the at least one stabilizing member is in the second position, the helical distal end at least partially protrudes from the second aperture in a helical form whereby it reversibly engages a tissue at or near the distal end of the needle.

11. The needle device of claim 10, further comprising:
a hub having a needle port, an injection port, a stabilizer port and an actuator, wherein a proximal end of the needle is connected to the needle port, the hub being configured to allow fluid communication between the injection port, the needle port and the first lumen; and
wherein a proximal end of the at least one stabilizing member is positioned in the stabilizer port and operably connected to the actuator, the actuator being configured such that the actuator can be moved longitudinally, thereby causing the at least one stabilizing member to slide longitudinally within the stabilizer port.

12. The needle device of claim 11, further comprising an elongated, flexible extension tube having a distal end connected to the injection port, and a proximal end configured to engage with a syringe.

13. The needle device of claim 10, wherein the at least one stabilizing member is made from nitinol.

14. The needle device of claim 10, wherein the distal end of the needle is configured to be echogenic.

15. The needle device of claim 10, wherein the at least one stabilizing member is made from round or flat wire.

16. The needle device of claim 15, wherein the stabilizing member is made from flat wire.

17. The needle device of claim 10, wherein the helical distal end of the at least one stabilizing member is configured such that when the helical distal end at least partially protrudes from the needle aperture, the sharp distal tip is offset from a longitudinal axis of the needle.

18. The needle device of claim 10, wherein the at least one stabilizing member comprises two or more stabilizing members.
